(19) United States
(12) Patent Application Publication
(10) Pub. No.: US 2024/0417243 A1
(43) Pub. Date: Dec. 19, 2024

(54) INTEGRATED PRESSURE DIAPHRAGMS

(71) Applicant: EDWARDS LIFESCIENCES CORPORATION, Irvine, CA (US)

(72) Inventors: Alexander H. Siemons, Yorba Linda, CA (US); Gustavo Rios, Pasadena, CA (US)

(21) Appl. No.: 18/819,033

(22) Filed: Aug. 29, 2024

Related U.S. Application Data
(63) Continuation of application No. PCT/US2023/015424, filed on Mar. 16, 2023.
(60) Provisional application No. 63/321,556, filed on Mar. 18, 2022.

(51) Int. Cl. B81C 1/00; A61B 5/0215; C23C 14/04; C23C 14/14; C23C 14/24

(52) U.S. Cl. CPC B81C 1/00158; A61B 5/0215; C23C 14/042; C23C 14/14; C23C 14/24; A61B 2562/0247; A61B 2562/12; B81C 2201/0133; B81C 2201/0143

(57) ABSTRACT

A method of manufacturing an integrated, flexible diaphragm for a pressure sensor using a physical vapor deposition process (PVD), including: providing a mandrel form, the mandrel form including a body portion and a diaphragm portion, placing the mandrel form in a vacuum deposition chamber, depositing a plurality of base layers of a material over at least a portion of the body portion of the mandrel form surrounding the diaphragm portion, and depositing a skin layer over the diaphragm portion and over the deposited plurality of base layers around the diaphragm portion to form the integrated, flexible diaphragm.

### RELATED APPLICATION

This application is a continuation of International Patent Application No. PCT/US23/15424, filed Mar. 16, 2023 and entitled INTEGRATED PRESSURE DIAPHRAGM, which claims priority to U.S. Provisional Patent Application Ser. No. 63/321,556, filed on Mar. 18, 2022 and entitled INTEGRATED PRESSURE DIAPHRAGM.

### BACKGROUND

The present disclosure generally relates to the field of sensor devices including diaphragms that deflect in response to pressure changes. Mechanical sealing between such diaphragms and the associated housing can affect suitability of such devices for certain applications.

### SUMMARY

Described herein are methods for forming deflectable diaphragms that inegrate with sensor housing/enclosure structures and/or layers. In particular, various pressure sensor packaging solutions are disclosed that provide for deposition of layer(s) of metal or other material over substrate structure(s) to form integrated diaphragm layer(s).

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 is a graph showing relationships between sensor diaphragm thickness, surface area, and sensitivity in accordance with one or more examples.

FIG. 2 is a block diagram showing a deposition system in accordance with one or more examples.

FIGS. 3A, 3B, and 3C provide a flow diagram illustrating a process for forming a pressure sensor diaphragm in accordance with one or more examples.

FIGS. 4A, 4B, and 4C provide images of pressure sensor diaphragm components corresponding to operations of the process of FIGS. 3A, 3B, and 3C according to one or more examples.

FIGS. 5A, 5B, and 5C provide a flow diagram illustrating a process for forming a pressure sensor diaphragm in accordance with one or more examples.

FIGS. 6A, 6B, 6C, 6D, 6E, and 6F provide images of forming a pressure sensor diaphragm corresponding to operations of the process of FIGS. 5A, 5B, and 5C according to one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to various examples. Although certain spatially relative terms, such as “outer,” “inner,” “upper,” “lower,” “below.” “above,” “vertical,” “horizontal,” “top,” “bottom,” “distal,” “proximal,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

The present disclosure relates to systems, devices, and methods for packaging devices configured for sensing one or more physiological parameters of a patient (e.g., blood pressure). Such pressure sensing/monitoring may be performed using cardiac implant devices having integrated pressure sensor diaphragms and/or associated components. Such devices can advantageously be packaged for long-term implantation in the cardiac environment. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

As described in detail below, implantable pressure sensors can be used to measure pressure levels. However, due to the accessibility and environmental conditions, only certain types of sensors and sensor packagings may be suitable for implantation for a given application. Examples of the present disclosure relate to the packaging of pressure sensor implant devices including certain electronics and telemetry features to allow for data and/or power communication wirelessly between the implanted sensor devices and one or more devices or systems external to the patient.

Aspects of the present disclosure relate to sensor devices comprising deflectable diaphragm components. In particular, inventive features disclosed herein can be implemented in the context of implantable sensor devices, wherein integrated diaphragm features can advantageously provide biocompatible sealing and/or encapsulation of internal sensor components.

With respect to implantable pressure sensor devices, anatomical considerations can necessitate the use of sensor devices having relatively small form factors. For example, it may be desirable to implant sensor devices, such as pressure sensor devices, using transcatheter procedures, wherein the sensor device is advanced to the target implantation site through one or more venous or arterial blood vessels and/or various tortuous access paths. Examples of the present disclosure advantageously can be implemented in sensor devices having a sufficiently small profile/size to be transported by and/or within a catheter, sheath, or other instrument configured for transcatheter access/use. In addition to sizing constraints associated with implantable sensor devices (e.g., pressure sensor devices), sensor sensitivity and/or dynamic range requirements or desires likewise may drive sensor design. For example, with respect to pressure sensor devices, deflectable pressure diaphragms associated with such devices may be designed in a manner as to provide sufficient sensitivity to pressure conditions to which the device is exposed.

Furthermore, implantable sensor devices, such as diaphragm-equipped pressure sensor devices, may further need to provide biocompatibility and/or encapsulation characteristics suitable for in vivo implantation. For example, with respect to implantation within certain anatomy, such as within a chamber of a heart, or other fluid-filled anatomical vessel/chamber, such environments can present certain pressure, turbulence, and corrosion conditions, which may be associated with fluid/blood characteristics and/or cardiac cycling. Relative to non-implant environments, the human body represents a very harsh environment for electrical implant devices. Examples of the present disclosure provide sensor implant devices that provide extended-duration and/or lifetime hermetic seals/sealing, which can be advantageous and/or critical for various reasons. For example, such hermetic scaling can prevent components of the environmental blood from degrading or otherwise interfering with the sensor and associated electronics. In addition, hermetic sealing of examples of the present disclosure can help prevent any non-biocompatible components of or associated with the sensor implant device from creating/causing toxic conditions within the body. Examples of the present disclosure furthermore provide sensor implant devices with ion-deposited shell components that are relatively thin and provide hermitically-sealed, integrated sensor diaphragms. Such designs can have a reduced package thickness compared to certain other welded-metal solutions, which may include interfaces between metal and/or ceramic components thereof that occupy undesirable amounts of space. By integrating multiple functional components into a single thin shell, the total package thickness/size can be minimized.

### Pressure Sensor Devices

Pressure sensors can incorporate deformable membranes that are used to measure pressure-induced deflection thereof, wherein the degree of deflection of the membrane is indicative of pressure conditions to which the sensor membrane is exposed at the implant location.

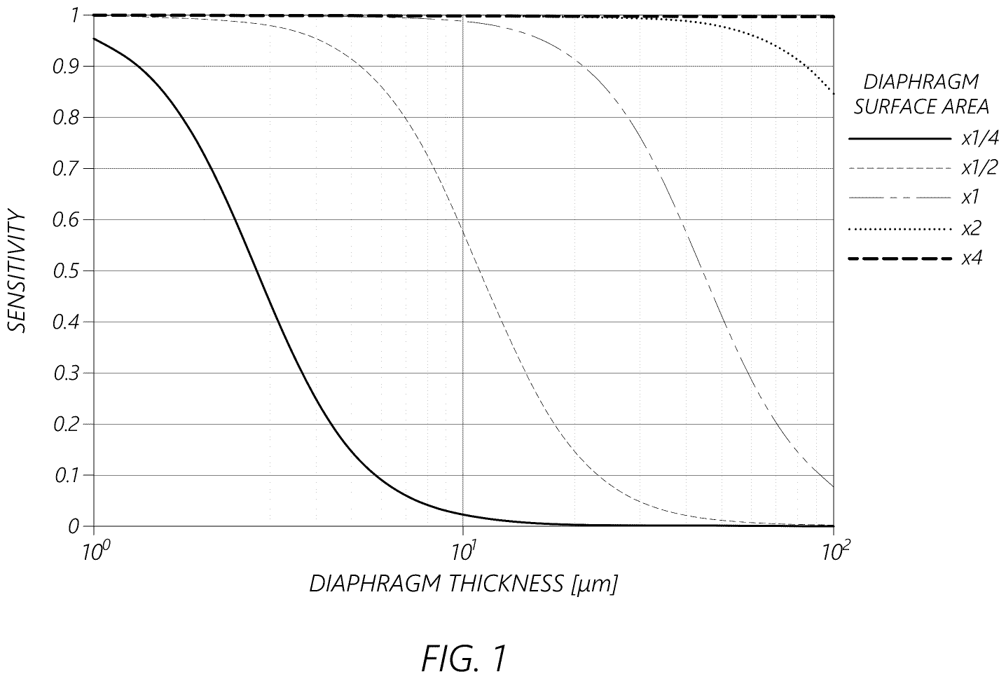

As referenced above, due to size constraints associated with implantable sensor devices, the area available for diaphragm components may likewise be constrained, depending on the design of the sensor device. For example, elongated sensor devices suitable for transcatheter transportation may generally be limited in profile/dimension with respect to the diameter/width thereof, whereas the length of the sensor device may be less constrained in some instances. Therefore, as sensor device profile is reduced, axial diaphragm area may likewise be reduced. As a diaphragm effective areas reduce, it may be necessary to reduce the thickness of the diaphragm as well in order to maintain sufficient sensitivity in the diaphragm. For example, FIG. 1 is a graph showing relationships between sensor diaphragm thickness, surface area, and sensitivity in accordance with one or more examples. As demonstrated in the graph of FIG. 1, diaphragms having relatively smaller surface area generally must be relatively thinner in order to achieve comparable sensitivity compared to diaphragms having relatively greater surface area and otherwise similar design.

Processes implemented to form thin foils can cause variable strain hardening of the formed materials, which may result in relatively large variation in mechanical performance of a formed diaphragm. Furthermore, relatively thin materials utilized for diaphragm formation can require careful handling and assembly processes to weld the diaphragm structure to the larger sealed body. At such scale, the processes implemented can influence the material properties and mechanics of the diaphragm, thereby causing additional variation in diaphragm performance.

In addition, certain sensor devices are designed with sensor diaphragms positioned/disposed at a distal end of the sensor device assembly. Since it can be advantageous to increase the area of the diaphragm to provide desirable sensitivity, as indicated in FIG. 1, increase in diaphragm area for such designs can be at the cost of increasing sensor device diameter/profile, potentially interfering with the ability to fit within a tubular catheter/shaft for delivery, particularly in consideration of diaphragm material thicknesses and deflection sensitivity according to the relationships demonstrated in the graph of FIG. 1. As sensor device designs evolve toward smaller and smaller-profile devices (e.g., millimeter-scale integrated implant devices), the ability to form and integrate such sensor device assemblies can become untenable with respect to certain design paradigms.

Examples of the present disclosure advantageously provide solutions for implantable pressure sensor devices that are relatively small in size, while providing sufficient and/or improved sensor performance/sensitivity by incorporating sensor diaphragms that have sufficiently large area and/or material thickness characteristics to provide such sensitivity. Such examples further provide, in some cases, suitable and/or improved biocompatibility characteristics and/or relatively simplified manufacturing processes.

Inventive sensor implant device solutions presented herein can achieve these benefits at least in part through integration of pressure sensor diaphragm components with other structural/mechanical housing/encapsulation component(s) of the device in one or more uniform and integrated layers of deposited material. For example, while certain pressure sensor devices require manufacturing processes that involve multi-part and/or multiple-process manufacturing to seal/mechanically-couple diaphragm components to other structural components, examples of the present disclosure allow for manufacturing without the need for such sealing/coupling step(s)/process(es). Furthermore, in addition to the advantages associated with manufacturing structural and diaphragm components of a sensor device as a single integrated/net component in relatively fewer manufacturing steps/processes, such devices in accordance with aspects of the present disclosure can provide superior mechanical properties relative to certain non-integrated diaphragm solutions. Furthermore, integration of diaphragm and shell components can greatly reduce component count and process steps required to produce the resulting sensor implant device and/or associated packaging. With fewer components and areas requiring hermetic sealing, more robust protective shells can be produced that present a reduced risk of failure/leakage. In addition, pressure sensor devices including integrated diaphragm components, as described in detail herein, can advantageously integrate and improve the consistency and/or transmission performance of sensor membranes/diaphragms that separates the biological environment from the internal sensor components of the sensor device.

Certain examples of the present disclosure provide alternatives to wrought-metal machining, stamping, grinding, or the like, of sensor diaphragms in order to provide diaphragms with reduced thicknesses and improved sensitivity. Such diaphragms may be advantageously formed using an ionized deposition process, rather than through stamping, welding, or other more complicated and/or inconsistent/error-prone processes. Furthermore, deposition of diaphragms in an integrated manner with sensor housing/shell structure can provide improved hermetic sealing, while also involving reduced risk of error due to reduced process variations. In some examples, diaphragms deposited/formed in accordance with aspects of the present disclosure comprise nitinol metal alloy rather than titanium, which may be utilized in other sensor designs.

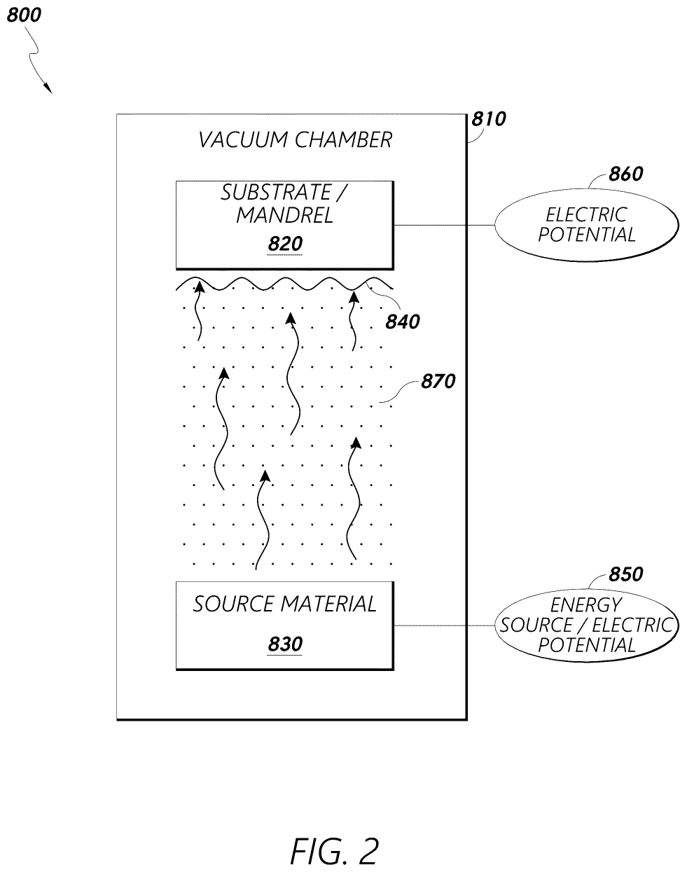

As referenced above, sensor diaphragms in accordance with aspects of the present disclosure may be manufactured/formed using ionized metal vapor deposition in some implementations. FIG. 2 is a block diagram showing a vacuum deposition system 800 in accordance with one or more examples. Physical vapor deposition (PVD) and other vacuum deposition processes can be used to produce relatively thin films and coatings. In the system 800, a source material 830 (e.g., metal) transitions from a condensed phase to a vapor phase 870 and then back to a thin film condensed phase 840. Sputtering or evaporation may be implemented to produce the vaporized/plasma gas 870. The plasma gas 870 is deposited on a substrate 820 to form the layer 840 of the deposited source material. The vacuum chamber 810 may advantageously be devoid of air and particles that could otherwise interfere with the directed deposition onto the substrate 820.

Transformation from solid 830 to gas 870 can be achieved through application of energy from an energy source 850. The energy source 850 may be any type of energy, including heat/thermal current, electrical current, and/or voltage potential relative to the potential 860 associated with the substrate 820. Energy may energize the source material 830 to produce the plasma form 870. The electric potential 860 relative to the source material 830 may serve to create a direction of the deposition flow 870 towards the substrate 820. Source material 830 may be positively charged in some cases, whereas the electric potential 860 of the substrate 820 may be negatively charged.

With respect to the various processes and devices disclosed herein, any type of deposition process may be implemented to produce the inventive shell housing components having integrated diaphragm layer(s). Examples may include, cathodic arc deposition, in which a high-power electric arc is discharged at the target (source) material to blast away some into highly ionized vapor to be deposited onto the workpiece. For electron-beam physical vapor deposition implementations, the material to be deposited is heated to a relatively high vapor pressure by electron bombardment in a vacuum and is transported by diffusion to be deposited by condensation onto a relatively cooler workpiece. For evaporative deposition, the material to be deposited may be heated to a relatively high vapor pressure by electrical resistance heating in a vacuum. As another example, close-space sublimation can involve placing the source material and substrate in relatively close proximity to one another and radiatively heated. Pulsed laser deposition may be implemented by ablating the source material into a vapor using a high-power laser. Pulsed electron deposition may be implemented by ablating the source material to generate a plasma under nonequilibrium conditions using a highly energetic pulsed electron beam.

In some example examples, sputter deposition may be implemented, wherein a glow plasma discharge, which may be localized around the target substrate by a magnet, bombards the source material, thereby sputtering some away as a vapor for subsequent deposition. For sputtering applications, a magnetron may be employed that utilize strong electric and magnetic fields to confine charged plasma particles close to the surface of the sputter target. Generally, in a magnetic field, electrons follow helical paths around magnetic field lines, undergoing more ionizing collisions with gaseous neutrals near the target surface than would otherwise occur. The extra ions of the sputter gas created as a result of these collisions can lead to a higher deposition rate. The plasma can also be sustained at a lower pressure this way. The sputtered atoms are neutrally charged and so are unaffected by the magnetic trap. Other sputtering techniques that can be implemented include ion-beam sputtering, reactive sputtering, ion-assisted deposition, high-power impulse magnetron sputtering, gas flow sputtering, or the like.

The substrate/mandrel 820 may be configured to rotate to allow for formation of a three-dimensional form/shell. The rotation may be controlled in a manner as to produce a relatively even distribution of deposition material over the mandrel 820 around the three-dimensional form.

Sensor implant devices having integrated and/or transverse-facing diaphragms in accordance with aspect of the present disclosure may include a housing comprising a shell formed of one or more layers of vapor deposition. For example, such layered deposition can involve depositing a thin layer of deposition material, followed by masking a portion of the deposition (e.g., a diaphragm portion) and subsequently depositing another, possibly thicker, layer. Alternatively, a thicker layer may be deposited over a masked diaphragm (or the diaphragm can subsequently be removed), after which, with no mask over the diaphragm, a relatively thinner layer may be applied to produce the integrated diaphragm and shell structure.

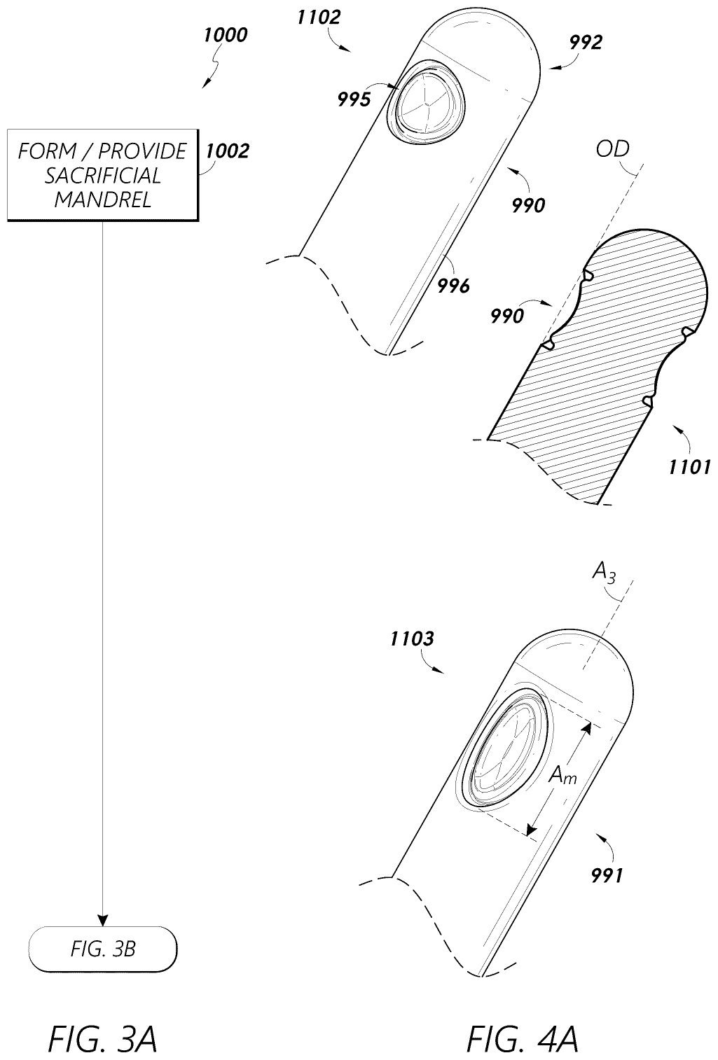

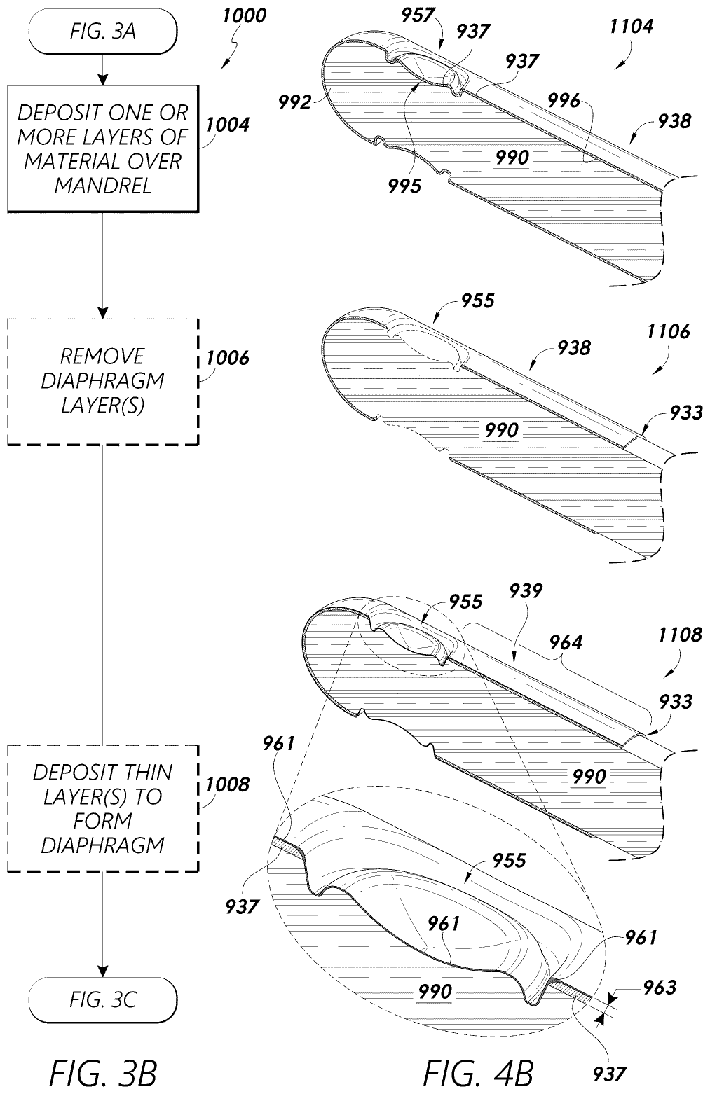

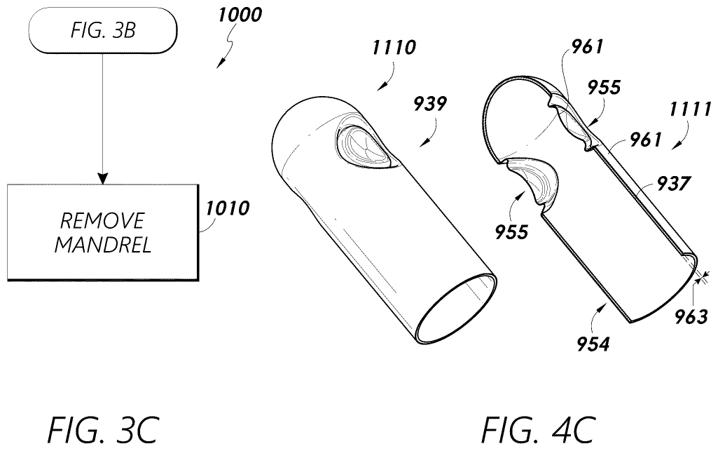

FIG. 3F show views of shells of sensor devices having one or more integrated diaphragm(s) in accordance with various examples. Some of the description below of sensor-covering shell/wall examples is described with reference to components shown in FIGS. 4A, 4B, and/or 4C; additionally, the description of the wall/shell 939 and associated features/components can be understood to refer to, or describe, the wall/shell 639 shown in FIGS. 6A-6F, or any other example disclosed herein. With reference to FIGS. 4A-4C (or any other figure of the present disclosure), the diaphragm(s) 955 and at least a portion of the associated shell/encasement 939 may be formed using a vapor deposition process in one or more stages.

The diaphragm(s) 955 may have a corrugated form, as shown, or may have any other form. The corrugated topology of the diaphragm 955 may facilitate deflection of the diaphragm 955 in a manner as to translate pressure to an internal sensor device (not shown or described in detail herein). The shell/wall 939 can be sealed to a base housing to enclose a cavity or chamber within the shell/wall 939.

The diaphragm(s) 955 may comprise a thin layer of deposited metal, such as nitinol, titanium, or other metal. The relatively thin layer of diaphragm 955 may be a unitary form with at least a portion of the body/sidewall portion(s) of the sensor enclosure wall 939. By integrating the diaphragm 955 with the wall 939, the diaphragm 955 can have an inherent seal with the shell structure, thereby providing increased protection for the internal components and/or protection from biological breakdown over time. The diaphragm(s) 955 provide the transmission window through which external pressure conditions are transferred. The diaphragm(s) 955 may be formed of a final deposition layer that provides an exterior skin over the wall 939, which provides structure for holding internal components within an assembled sensor device.

Where the exterior layer(s) of the encapsulation wall 939 is formed of deposited metal deposited in unitary form, such deposition can provide structural continuity between the diaphragm(s) 955 and the body of the wall 939. Such continuity can be advantageous for various reasons. In examples in which separate metal components/layers form the diaphragm and the structural housing, slight differences in the metal structure and/or interfaces therebetween can introduce imperfections and/or structural defects in the overall structure of the housing. Therefore forming such components of a common source material and through a common deposition application can provide structural benefits for the wall 939.

The structural wall 939 may be formed of one or more coats, layers, or processes, such that the body portion may have a different thickness than certain other portions, such as the diaphragm 955. For example, in some implementations, an initial relatively thicker layer 937 (e.g. approximately 100-300 μm; 250 μm) may be deposited on a substrate to form the relatively thicker and/or more rigid body of the wall 939 outside of the diaphragm area 955. For example, such layer(s) may be deposited/formed while the diaphragm area 955 is masked. Alternatively, the thicker coating/deposition of metal (e.g., nitinol) may be deposited over the entire wall 939, including the diaphragm portion(s) 955, wherein a thickness of the deposited material on the diaphragm(s) may be etched away or otherwise thinned to produce a relatively thinner diaphragm, which may be desirable with respect to sensitivity/flexibility of the diaphragm. In some examples, the diaphragm thickness may be approximately 10 μm, such that the diaphragm is approximately 3-15% (e.g., 4%, 5%, 10%, or any number therebetween) as thick as the structure of the wall 939 outside of the diaphragm areas (i.e., in the body portion of the wall 939). For example, where the diaphragm 955 is masked during deposition of the thicker layer 937 of the wall 939, after such deposition, the mask may be removed to allow for deposition of the same metal/material over the entirety of the wall 939, including over the portions of the diaphragm(s) and area immediately adjacent thereto, wherein such subsequent layering/deposition may be relatively thin skin/layer (e.g., approximately 10 μm thick, or between 10-40 μm thick).

In some implementations, the thin-layer diaphragm may be deposited over a mandrel/substrate surface 990, after which, the diaphragm 955 may be masked to allow for subsequent deposition of the thicker layer 937 over the areas of the mandrel/substrate and thin deposition layer outside of the diaphragm area 955 to fully form the wall 939. Removal of the diaphragm mask and the mandrel/substrate to produce the complete integrated shell and diaphragm.

A deposition process may be implemented to deposit a thickness/layer of metal or other material (e.g., nitinol) over a mandrel or other substrate 990, wherein a laser process may be implemented to selectively remove at least a portion of such deposition in the area of the diaphragm(s) 955. For example, in some limitations, substantially all of the deposited material in the area of the diaphragm(s) may be removed. After such diaphragm removal, the structure may be placed back into the deposition chamber (e.g., PVD vacuum chamber), wherein a final skin/layer may be applied over the previously deposited material that has not been removed, as well as over the diaphragm area where the previous deposition was removed/thinned or blocked.

In some implementations, masking of the diaphragm may be implemented by using a resist layer over the diaphragm(s) to prevent buildup of the deposited material in the covered area (e.g., a sacrificial layer). By depositing a final integrated layer/coating over the structure including the diaphragms, a robust hermetic seal may be produced along the entire wall 939. That is, the process of applying first a thick film layer over the body of the shell followed by a final thin film coating over the diaphragms and the body of the shell, including the area surrounding the diaphragm(s), can provide desirable adhesion between the thin outer skin/diaphragm layer and the remaining structure, provided that the surface of the structure is maintained relatively clean between process stages and the same deposition material is applied in both stages. In instances where contamination is permitted between process steps, adhesion may be compromised, although in some instances the structural integrity may be sufficient to meet the particular needs. The deposition material used to form the layer(s) of the wall 939 include nitinol, titanium, stainless steel, and other metals having similar properties. A base housing component, which can be coupled to the wall 939 to form the enclosed housing container, can include zirconium, ceramic, or other material having similar properties. The base housing can be bonded or brazed to the wall 939 in a manner as to maintain a hermetic seal between the two components of the device.

The diaphragm(s) 955 may have any suitable or desirable shape or form, including corrugated diaphragms, dimpled/depression diaphragms, flat diaphragms (e.g., flat along the curvature of the cylindrical form of the wall 939, or diametrically flat across an arc segment of the cylinder body. For example, the diaphragm(s) may be formed of a layer of material that is deposited on and lies in a plane that is tangential to the circumference/perimeter of the cylinder, which is sunk radially within the outer diameter of the cylinder.

FIGS. 3A-3C (collectively referred to as FIG. 3 in some contexts) provide a flow diagram illustrating a process 1000 for packaging a sensor device in accordance with one or more examples. FIGS. 4A-4C (collectively referred to as FIG. 4 in some contexts) provide images of pressure sensor packaging corresponding to operations of the process of FIGS. 3A-3C according to one or more examples.

At block 1002, the process 1000 involves forming or providing a sacrificial mandrel 990 for formation of a sensor-encapsulation shell/wall as described in detail herein. For example, the sacrificial mandrel 990, shown in image 1102 of FIG. 4A, can comprise material configured to be selectively etched away after deposition on an outer surface thereof of the relevant process material (e.g., such as an outer sacrificial layer). The mandrel 990 may comprise stainless steel in some examples. The use of an electrically conductive material may be desirable to facilitate direction of deposition vapor in a vacuum chamber, which may be reliant upon implementation of an electric potential between the source material and the mandrel substrate.

The mandrel 990 may be primarily cylindrical in shape, and may be solid or hollow. Examples incorporating hollow mandrels may be advantageous as requiring less sacrificial material etching/removal when deposition is complete. The mandrel 990 may comprise one or more sacrificial layers. The mandrel 990 may include a dome-shaped and/or flat endcap portion 992. That is, although a semi-sphere dome-type form is shown as the endcap portion 992 in image 1102, it should be understood that the end portion 992 may have any suitable or desirable shape. Furthermore, although the mandrel 990 is shown as having a cylindrical shape, in some implementations, the mandrel (and resulting sensor shell deposition) as a rectangular or elliptical/oval shape.

The mandrel 990 can include diaphragm portion(s) 995 having shape/form corresponding to a desired diaphragm shape or form, such that deposition of material over such areas can produce the desired diaphragm shape/form for the sensor shell when the conformal deposition is applied to the surface thereof. The mandrel 990 can be planar in at least an area surrounding diaphragm portion(s) 995. Although examples are disclosed herein in the context of sensor devices including substantially circular diaphragms, it should be understood that any shape of the diaphragm may be implemented, including elliptical diaphragms, as shown in image 1103 of FIG. 4A with respect to the illustrated alternative mandrel 991. In the example of image 1103, a major axis of the diaphragm is parallel to the longitudinal axis of the shell. As shown from the cross-sectional side view of image 1101 of FIG. 4A, the diaphragm 995, as defined by the form of the mandrel 990, may be stepped-back from the outer diameter of the cylinder to provide protection for the diaphragm from the implant environment and/or structural damage associated with manufacturing, delivery, and maintenance in vivo.

Although a three-dimensional mandrel form is illustrated and described, wherein the shell is formed by depositing a three-dimensional cylindrical form over the mandrel, it should be understood that examples of the present disclosure may implement two-dimensional deposition of a sheet of material shaped and configured for post-deposition forming of the three-dimensional shell from the sheet of deposited material. Therefore, any example disclosed herein may be understood to comprise a cylindrical sensor shell deposition or deposition of a sheet that is later formed into a sensor shell. However, forming a shell from a sheet of material can introduce complexity in the manufacturing process and complicate the sealing of the shell for biocompatibility purposes.

At block 1004, the process 1000 involves depositing one or more layers of material over the mandrel 990, as shown in image 1104 of FIG. 4B. For example, such layer(s) may be applied over the exterior surface of the mandrel 990 using physical vapor deposition or other deposition or sputtering process, as described in detail above. The deposition material may be any suitable or desirable material compatible with deposition processes as disclosed herein. For example, the deposited layer(s) of material 937 may comprise nitinol, titanium, and/or other metal.

The operation(s) associated with block 1004 may involve using physical vapor deposition to form a shell of material over the sacrificial mandrel 990 and/or over at least a portion thereof. The target material may be applied to the mandrel 990 in one or more layers to create a relatively even deposition of material 937 over the surface of the mandrel 990, such as over substantially the entire surface thereof with respect to one or more perspectives or angles of the mandrel. In some implementations, the initial deposition of layer(s) of shell material is applied over the diaphragm portion 995 of the mandrel 990 as well as the sidewall 996 and/or end 992 portions. The initial layer(s) 937 of deposition material may be approximately 100-300 μm in thickness such as between approximately 150-250 μm in some implementations. For example, the thickness may be between about 150-180 μm in some implementations.

A particular deposition process implemented may utilize sputtering or evaporation to generate a vapor in the form of molecules or ions from a target source material. Such vapor may be transported and deposited onto the exterior surface of the sacrificial mandrel 990 to create a coating 937. The process of deposition may continue until the desired thickness is achieved. In some implementations, planer magnetron physical vapor deposition is implemented to deposit the layer(s) 937 on the mandrel 990. Such processes can advantageously produce relatively flat application layers. Furthermore, inverted cylindrical magnetron deposition processes can advantageously allow for deposition to occur over a three-dimensional cylindrical surface, as is the mandrel 990 illustrated and described in connection with FIGS. 3 and 4. Deposition layer(s) created by such processes can produce relatively more pristine and isotropic layers compared to wrought forms/materials that are cut, machined, ground, or stamped into the shell form having a diaphragm as described herein.

At block 1006, the process 1000 may involve removing one or more layers of material in the area of the diaphragm(s) 955 to completely remove the diaphragm portion(s) 957 of the wall 939 formed from the initial deposition layer(s) 937, or at least some of the thickness thereof, to produce a relatively thin diaphragm deposition, or completely remove the deposition, over the diaphragm portion(s) 995 of the mandrel 990.

In some implementations, a femto-second laser is utilized to mill-out the diaphragm area 957 of the shell layer(s) 937. Such milling may be performed in any suitable or desirable manner. For example, the diaphragm portion 957 of the shell layer(s) 937 may be completely removed, thereby exposing the diaphragm portion 995 of the mandrel 990 beneath. Alternatively, only a portion of the thickness of the diaphragm portion 957 of the shell layer(s) 937 may be removed in some implementations, or selective areas or thicknesses of the deposited shell layer(s) 937 may be removed, wherein certain areas of the diaphragm portion 957 may be removed down to the substrate 990, while some deposited material is selectively left behind in the diaphragm area 957, thereby allowing/providing a relatively high degree of customization with respect to the mechanics of the final diaphragm component. The material removal associated with block 1006 can advantageously involve removing material that follows the contours/topology of the diaphragm portion 995 of the mandrel 990, whatever shape is implemented therefore. The contours around the diaphragm 995 of the mandrel 990 may advantageously conform to minimum-angle requirements for even deposition according to the implemented deposition process.

Ultimately, the sensor wall 939 formed will include a diaphragm 955 that is thinner than the sidewalls 937 of the wall 939. The deposition material properties can also allow for relatively precise high-resolution laser milling and ablation, potentially allowing for sub-micron tolerances of the removed material.

In some implementations, the produced wall 939 with removed/missing diaphragm portions as shown in image 1106 of FIG. 4B may be produced in any suitable or desirable manner. For example, previously-deposited layer(s) 937 of the wall 939 over the diaphragm 995 may be removed or thinned, as described above. Alternatively, in some implementations, a masking structure or agent may be disposed/applied over the diaphragm region(s) 995 of the substrate 990 when the layer(s) 937 of the wall 939 are initially deposited. The mask may then be removed to expose the diaphragm portion(s) 995 of the mandrel 990 to allow for further deposition in such areas.

In implementations in which laser milling or other process is implemented to produce the desired thickness for the diaphragm 955, the formation of the final wall 939 may be complete following the operation(s) associated with block 1006. In some implementations, laser milling may further be utilized to remove the deposited material in the area of the open edge of the wall 939 to produce a proximal edge 933 that has a relatively clean axial line about the circumference of the mandrel 990.

In implementations in which the mandrel and wall 939 are removed from the vacuum deposition chamber for the purpose of laser etching or otherwise removing material in the area of the diaphragm 957, wherein further deposition layer(s) are intended for deposition on the wall 939, such removal and/or replacement from/in the vacuum chamber may negatively impact adhesion between the initially deposited layer(s) 937 and any subsequently-deposited layer(s). However, such detrimental effects may nevertheless be desirable as a means of achieving the integrated diaphragm structure with a relatively thin diaphragm as described in connection with the examples disclosed herein. The diaphragm area 957 may be selectively etched away in a manner as to avoid etching away portions of the sidewall and/or end portions of the wall 939.

In some implementations, the process 1000 involves, at block 1008, depositing one or more relatively thin layer(s) over the wall 939 and diaphragm portion(s) 995 of the mandrel 990 (e.g., in implementations in which the mandrel 995 is exposed in the diaphragm areas). Image 1108 of FIG. 4B shows an example including a thin layer 961 deposited over the wall 939 and diaphragm portion 995 of the mandrel 995.

The additional secondary layer(s) 961 applied over the wall 939 and diaphragm 995 can be for example, between 10-50 μm thick. Such thickness 961 may be combined with the thickness 937 of the end and sidewall portions to form a relatively thick 963 sidewall and endcap structure for the final wall 939, while producing a relatively thinner diaphragm 955, which may be between 3-20% as thick as the combined layer(s) 963 in the sidewall portions. The new deposited skin/layer(s) 961 can include the relatively thin diaphragm 955 integrated with a thin layer 961 that is laid over the previously-deposited shell 937/939 in the sidewall portions 964, wherein the new/outer layer/skin 961 bonds to the outer surface of the shell 937/939 to create a single/integrated body for the wall 939. The material used for the second deposition 961 associated with block 1008 may advantageously be the same deposition material as applied in the initial deposition process, which may improve adhesion and integration between the layers. The different layers of deposition may be bonded at the molecular level, thereby providing desirable integration. The diaphragm layer(s) may be electro-deposited in some implementations. Furthermore, the surfaces of such deposition may be configured to reduce tissue growth and adhesion thereon in some implementations.

In some implementations, as an initial deposition operation/step, a thin layer is deposited over the mandrel 990, including over the area of the diaphragm 995. After deposition of such thin layer over the diaphragm 995 and surrounding area(s) (e.g., end 992 and/or sidewall 996 portions), the diaphragm area 995/955 may be masked-off, such that the thin diaphragm layer 955 is protected from further deposition/layering. With the diaphragm 955 masked, additional layer(s) of deposition material may then be deposited over the mandrel and/or previously-deposited thin layer of shell. Such additional deposition may be relatively thicker than the thin diaphragm layer, thereby producing the relatively thick shell 937. The masking may then be removed, thereby revealing a relatively thin diaphragm layer 955 integrated with a relatively thicker shell structure 937. The operations described in this paragraph may be implemented in whole or in part as an alternative to, or in addition to, the operations shown in FIG. 3B, or as example implementations of various operations shown in FIG. 3B.

Although some examples are disclosed in which the primary 937 and secondary 961 deposition layer(s) comprise the same material (e.g., metal or metal alloy), in some implementations, the wall 939 may be formed of primary 937 and secondary 961 depositions that comprise different materials, wherein such layers may comprise materials selected to improve or tune the mechanical properties of the wall 939. For example, the different layers may include an outer layer 961 designed to provide suitable biocompatibility properties, corrosion resistance, super elasticity, or other feature(s), whereas the internal layer(s) 937 may be designed to provide desired structural rigidity/strength for the wall 939. The deposited wall 939 provides a unique design with transverse/radial diaphragms.

At block 1010, the process 1000 involves removing the sacrificial mandrel 990. For example, the mandrel substrate (e.g., stainless steel, copper, or the like) may be removed from the completed deposited shell/form 939 to leave the hollow shell. With the internal mandrel etched-out, the complete net form of the wall 939 may be left with a relatively thick structural body and a relatively thinned/thin diaphragm 955. Removing the mandrel can involve dissolving the mandrel form using any suitable or desirable type of etchant.

When the mandrel 990 is etched away, the complete net shell form 939 is isolated, wherein the wall 939 includes a structural body configured for connection to base component of a sensor device housing, as described herein. The wall 939 includes a tuned thin layer 961 in the diaphragm area 955 to provide desirable pressure transduction functionality, wherein the diaphragm 955 is substantially integrated at the molecular level with the body of the wall 939 without welding, brazing, or other bonding method, which, for reasons described above, can introduce undesired complexity and/or risk of error into the process.

The image 1110 in FIG. 4C shows the formed wall 939 with the sacrificial mandrel removed from therein. As apparent in the cross-sectional view of image 1111, the wall 939 is substantially hollow after removal of the mandrel. The cross-sectional image 1111 shows the relatively thicker body portion 954 integrated with the thinner diaphragm portion 955.

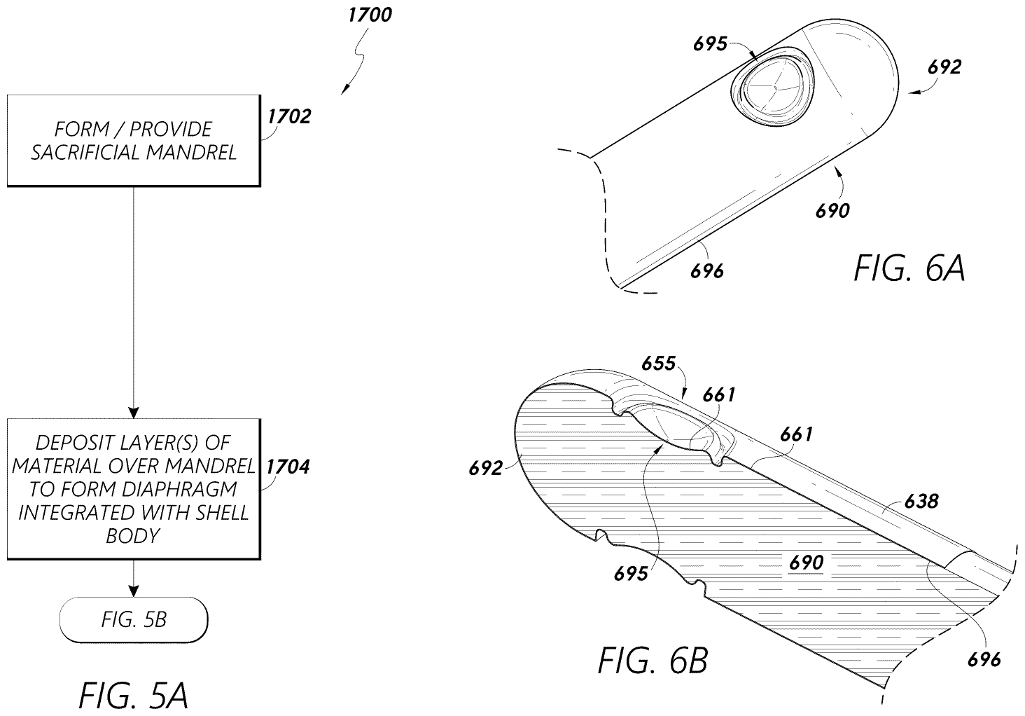

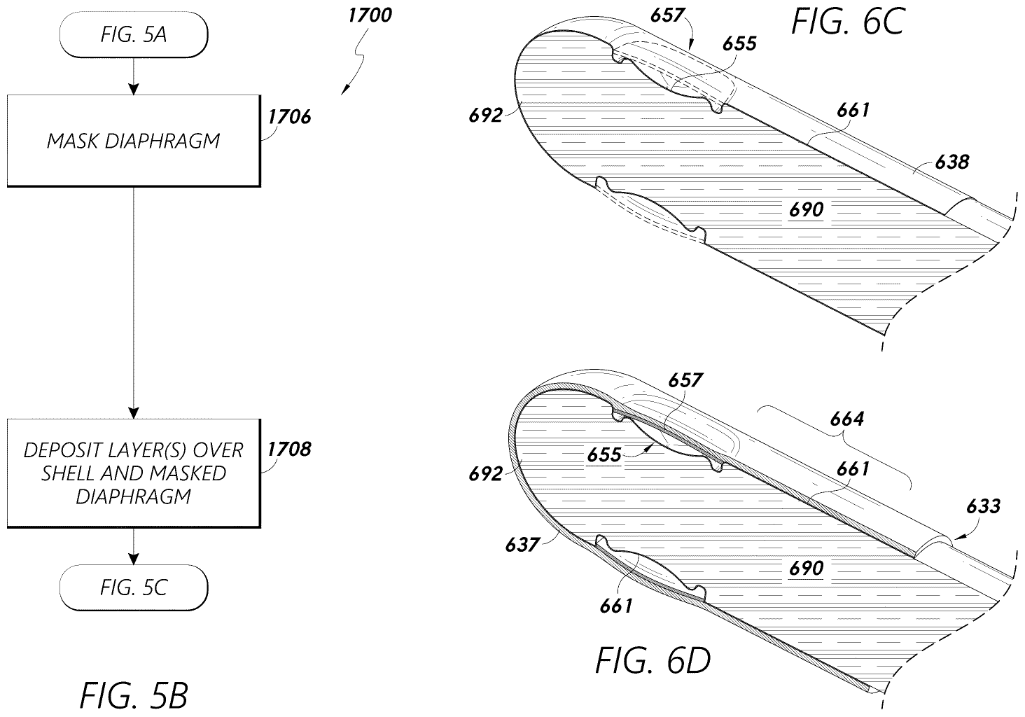

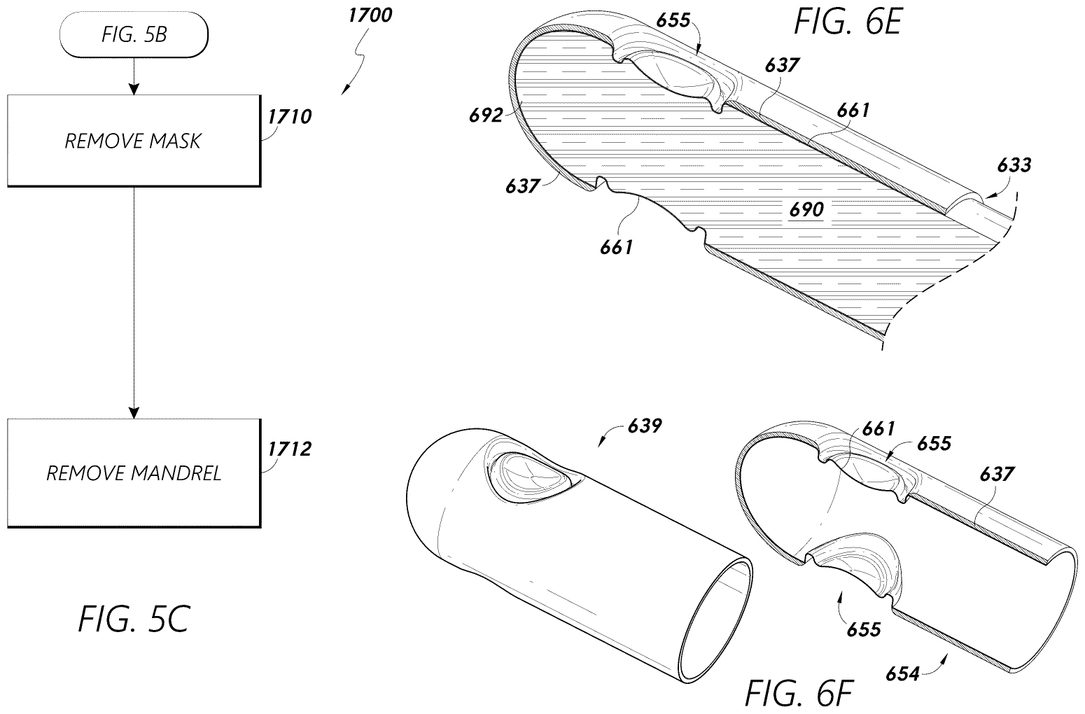

FIGS. 5A-5C (collectively referred to as FIG. 5 in some contexts) provide a flow diagram illustrating a process for packaging a sensor device in accordance with one or more examples. FIGS. 6A-6F (collectively referred to as FIG. 6 in some contexts) provide images of pressure sensor packaging components corresponding to operations of the process of FIGS. 5A-5C according to one or more examples.

At block 1702, the process 1700 involves forming or providing a sacrificial mandrel 690 for formation of a sensor shell as described in detail herein. For example, the sacrificial mandrel 690, shown in FIG. 6A, can comprise material configured to be selectively etched away after deposition on an outer surface thereof of the relevant process material. The mandrel 690 may comprise stainless steel in some examples. The use of an electrically conductive material may be desirable to facilitate direction of deposition vapor in a vacuum chamber, which may be reliant upon implementation of an electric potential between the source material and the mandrel substrate.

The mandrel 690 may be cylindrical or planar in shape, and may be solid or hollow. Examples incorporating hollow mandrels may be advantageous as requiring less sacrificial material etching/removal when deposition is complete. The mandrel 690 may include a dome-shaped and/or flat endcap portion 692. That is, although a semi-sphere dome-type form is shown as the endcap portion 692 in FIG. 6A, it should be understood that the end portion 692 may have any suitable or desirable shape. Furthermore, although the mandrel 690 is shown as having a cylindrical shape, in some implementations, the mandrel (and resulting sensor shell deposition) as a rectangular or elliptical/oval shape.

The mandrel 690 can include diaphragm portion(s) 695 having a shape/form corresponding to a desired pressure diaphragm shape or form, such that deposition of material over such areas can produce the desired diaphragm shape/form for the sensor shell when the conformal deposition is applied to the surface thereof. Although examples are disclosed herein in the context of sensor devices including substantially circular diaphragms, it should be understood that any shape of the diaphragm may be implemented, including elliptical diaphragms.

Although a three-dimensional mandrel form is illustrated and described, wherein the shell is formed by depositing a three-dimensional cylindrical form over the mandrel, it should be understood that examples of the present disclosure may implement two-dimensional deposition of a sheet of material shaped and configured for post-deposition forming of the three-dimensional shell from the sheet of deposited material. Therefore, any example disclosed herein may be understood to comprise a cylindrical sensor shell deposition or deposition of a sheet that is later formed into a sensor shell. However, forming a shell from a sheet of material can introduce complexity in the manufacturing process and complicate the sealing of the shell for biocompatibility purposes.

At block 1704, the process 1700 involves depositing one or more layers 661 of material over the mandrel 690. For example, such layer(s) 661 may be applied over the exterior surface of the mandrel 690 using physical vapor deposition or other deposition or sputtering process, as described in detail above. The deposition material may be any suitable or desirable material compatible with deposition processes as disclosed herein. For example, the deposited layer(s) of material 661 may comprise nitinol, titanium, and/or other metal.

The operation(s) associated with block 1704 may involve using physical vapor deposition to form a shell of material over the sacrificial mandrel 690 and/or over at least a portion thereof. The target material may be applied to the mandrel 690 in one or more layers to create a relatively even deposition of material 661 over the surface of the mandrel 690, such as over substantially the entire surface thereof with respect to one or more perspectives or angles of the mandrel. In some implementations, the initial deposition of layer(s) of shell material 661 is applied over the diaphragm portion 695 of the mandrel 690 to form a diaphragm layer 955, as well as the sidewall 696 and/or end 692 portions. The initial layer(s) 661 applied over the mandrel 690 can be relatively thin, such as, for example, between 10-50 μm thick. FIG. 6B shows the thin shell layer 661 deposited on the mandrel 690.

A particular deposition process implemented may utilize sputtering or evaporation to generate a vapor in the form of molecules or ions from a target source material. Such vapor may be transported and deposited onto the exterior surface of the sacrificial mandrel 690 to create a coating 661. The process of deposition may continue until the desired thickness is achieved. In some implementations, planer magnetron physical vapor deposition is implemented to deposit the layer(s) 661 on the mandrel 690. Such processes can advantageously produce relatively flat application layers. Furthermore, inverted cylindrical magnetron deposition processes can advantageously allow for deposition to occur over a three-dimensional cylindrical surface, as is the mandrel 690 illustrated and described in connection with FIGS. 5 and 6. Deposition layer(s) created by such processes can produce relatively more pristine and isotropic layers compared to wrought forms/materials that are cut, machined, ground, or stamped into the shell form having a diaphragm as described herein.

At block 1706, the process 1700 may involve masking the area of the deposited diaphragm(s) 655 with any suitable or desirable material to cover and protect the diaphragm layer(s) 655 from subsequent deposition(s). The diaphragm mask/cover 657 can be places/disposed over the diaphragm 655 using any type of application process known in the art or described herein. FIG. 6C shows the mask/cover 657 disposed over the diaphragm 655.

In implementations in which the mandrel 690 and shell 639 are removed from the vacuum deposition chamber for the purpose of masking material in the area of the diaphragm 655, wherein further deposition layer(s) are intended for deposition on the shell 639, such removal and/or replacement from/in the vacuum chamber may negatively impact adhesion between the initially deposited layer(s) 661 and any subsequently-deposited layer(s). However, such detrimental effects may nevertheless be desirable as a means of achieving the integrated diaphragm structure with a relatively thin diaphragm as described in connection with the examples disclosed herein. The diaphragm area 655 may be selectively masked in a manner as to avoid application of subsequent layer(s) onto the diaphragm layer(s) 655.

The process 1700 further involves, at block 1708, depositing one or more relatively thick secondary layer(s) 637 over the shell 639 and diaphragm mask(s) 657. The secondary layer(s) 637 of deposition material may be approximately 170-300 μm in thickness, such as between approximately 150-250 μm in some implementations. For example, the thickness may be between about 150-180 μm in some implementations. Such thickness 637 may be combined with the thickness 661 of the end and sidewall portions to form a relatively thick 664 sidewall and endcap structure for the final shell 639, while producing a relatively thinner diaphragm 655, which may be between 3-20% as thick as the combined layer(s) 664 in the sidewall portions. The new deposited(s) 637 can be laid over the mask 657 and the thin layer 661 in the sidewall portions 664, wherein the new/outer layer(s) 637 bond to the outer surface of the thinner shell 661 to create a single/integrated body for the shell 639. The material used for the second deposition 637 associated with block 1708 may advantageously be the same deposition material as applied in the initial deposition process, which may improve adhesion and integration between the layers. The different layers of deposition may be bonded at the molecular level, thereby providing desirable integration. Furthermore, the surfaces of such deposition may be configured to reduce tissue growth and adhesion thereon in some implementations. Although some examples are disclosed in which the primary 661 and secondary 637 deposition layer(s) comprise the same material (e.g., metal or metal alloy), in some implementations, the shell 639 may be formed of primary 661 and secondary 637 depositions that comprise different materials, wherein such layers may comprise materials selected to improve or tune the mechanical properties of the shell 639. FIG. 6D shows the thick layer 637 deposited over the shell 661 (in the area outside of the diaphragm) and the mask 657. In some implementations, laser milling may be utilized to remove the deposited material in the area of the open edge of the shell 639 to produce a proximal edge 633 that has a relatively clean axial line about the circumference of the mandrel 690.

At block 1710, the process 1700 may involve removing the masking/mask 657, thereby revealing the relatively thin diaphragm layer 655 integrated with a relatively thicker shell structure 637. In some implementations, removal of the mask 657 may allow for further deposition in such areas. FIG. 6E shows the mask 657 removed, exposing the thin-layer diaphragm 655.

At block 1712, the process 1700 involves removing the sacrificial mandrel 690. For example, the mandrel substrate (e.g., stainless steel, copper, or the like) may be removed from the completed deposited shell/wall 639 to leave the hollow shell. With the internal mandrel etched-out, the complete net form of the shell/wall 639 may be left with a relatively thick structural body and a relatively thinned/thin diaphragm 655. Removing the mandrel can involve dissolving the mandrel form using any suitable or desirable type of etchant. FIG. 6F shows the isolated shell/wall 639 with the mandrel 690 removed.

The isolated shell/wall 639 includes a structural body configured for connection to a base component of a sensor device housing. The shell/wall 639 includes a tuned thin layer 661 in the diaphragm area 655 to provide desirable pressure transduction functionality, wherein the diaphragm 655 is substantially integrated at the molecular level with the body of the shell/wall 639 without welding, brazing, or other bonding method, which, for reasons described above, can introduce undesired complexity and/or risk of error into the process. The shell/wall 639 may be substantially hollow after removal of the mandrel 690.

### Additional Description of Examples

Provided below is a list of examples, each of which may include aspects of any of the other examples disclosed herein. Furthermore, aspects of any example described above may be implemented in any of the numbered examples provided below.

Example 1: A method of manufacturing an integrated, flexible diaphragm, the method comprising: providing a mandrel form, the mandrel form including a body portion and a diaphragm portion; placing the mandrel form in a vacuum deposition chamber; depositing a plurality of base layers of a material over at least a portion of the body portion of the mandrel form surrounding the diaphragm portion; and depositing a skin layer over the diaphragm portion and over the deposited plurality of base layers around the diaphragm portion to form the integrated, flexible diaphragm.

Example 2: The method of any example herein, especially Example 1, further comprising prior to said depositing the skin layer, removing a portion of the plurality of base layers in an area of the diaphragm portion.

Example 3: The method of any example herein, especially Example 2, wherein said removing the portion of the plurality of base layers is performed using a laser.

Example 4: The method of any example herein, especially Example 1, further comprising: prior to said depositing the plurality of base layers of material, masking the diaphragm portion with a sacrificial layer.

Example 5: The method of any example herein, especially Example 1, further comprising: after said depositing the skin layer, removing the mandrel form from the one or more base layers and the skin layer.

Example 6: The method of any example herein, especially Example 5, wherein said removing the mandrel form involves dissolving the mandrel form using an etchant.

Example 7: The method of any example herein, especially Example 1, wherein the integrated, flexible diaphragm is flexible relative to the plurality of base layers.

Example 8: The method of any example herein, especially Example 1, wherein the integrated, flexible diaphragm has a thickness that is between 10-40 μm.

Example 9: The method of any example herein, especially Example 1, wherein the integrated, flexible diaphragm has a thickness that is approximately 3-15% of a combined thickness of the skin layer and the plurality of base layers.

Example 10: The method of any example herein, especially Example 1, wherein the integrated, flexible diaphragm comprises a plurality of corrugations.

Example 11: The method of any example herein, especially Example 10, wherein the plurality of corrugations comprise one or more concentric rings.

Example 12: The method of any example herein, especially Example 1, wherein the skin layer comprises a plurality of layers of the material.

Example 13: The method of any example herein, especially Example 1, wherein the skin layer comprises a superelastic nickel titanium material.

Example 14: The method of any example herein, especially Examples 1 and 13, wherein the material of the plurality of base layers comprises superelastic nickel titanium.

Example 15: The method of any example herein, especially Example 1, wherein the skin layer comprises a superelastic nickel titanium material.

Example 16: The method of any example herein, especially Example 1, wherein the portion of the body portion of the mandrel form surrounding the diaphragm portion is planar.

Example 17: The method of any example herein, especially Example 1, wherein the portion of the body portion of the mandrel form surrounding the diaphragm portion is curved.

Example 18: A method of manufacturing an integrated, flexible diaphragm, the method comprising: providing a mandrel form, the mandrel form including a body portion and a diaphragm portion; placing the mandrel form in a vacuum deposition chamber; depositing one or more first layers of material over the diaphragm portion and at least a portion of the body portion of the mandrel form surrounding the diaphragm portion to form the integrated, flexible diaphragm; disposing a mask layer over the diaphragm; depositing one or more second layers of material over the masked diaphragm and at least the portion of the body portion of the mandrel form surrounding the diaphragm portion.

Example 19: The method of any example herein, especially Example 18, further comprising: prior to said depositing the one or more first layers of material, masking the mandrel form with a sacrificial layer.

Example 20: The method of any example herein, especially Example 18, further comprising: after said depositing the one or more second layers of material, removing the mandrel form from the one or more first layers of material and the one or more second layers of material.

Example 21: The method of any example herein, especially Example 20, wherein said removing the mandrel form involves dissolving the mandrel form or one or more layers thereof using an etchant.

Example 22: The method of any example herein, especially Example 18, wherein the integrated, flexible diaphragm is flexible relative to the one or more second layers of material.

Example 23: The method of any example herein, especially Example 18, wherein the integrated, flexible diaphragm has a thickness that is between 10-40 μm.

Example 24: The method of any example herein, especially Example 18, wherein the integrated, flexible diaphragm has a thickness that is approximately 3-15% of a combined thickness of the one or more first layers of material and the one or more second layers of material.

Example 25: The method of any example herein, especially Example 18, wherein the integrated, flexible diaphragm comprises a plurality of corrugations.

Example 26: The method of any example herein, especially Example 25, wherein the plurality of corrugations comprise one or more concentric rings.

Example 27: The method of any example herein, especially Example 18, wherein the integrated, flexible diaphragm comprises a superelastic nickel titanium material.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including.” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of manufacturing an integrated, flexible diaphragm, the method comprising:
providing a mandrel form, the mandrel form including a body portion and a diaphragm portion;
placing the mandrel form in a vacuum deposition chamber;
depositing a plurality of base layers of a material over at least a portion of the body portion of the mandrel form surrounding the diaphragm portion; and
depositing a skin layer over the diaphragm portion and over the deposited plurality of base layers around the diaphragm portion to form the integrated, flexible diaphragm.

2. The method of claim 1, further comprising:
prior to said depositing the skin layer, removing a portion of the plurality of base layers in an area of the diaphragm portion.

3. The method of claim 2, wherein said removing the portion of the plurality of base layers is performed using a laser.

4. The method of claim 1, further comprising:
prior to said depositing the plurality of base layers of material, masking the diaphragm portion with a sacrificial layer.

5. The method of claim 1, further comprising:
after said depositing the skin layer, removing the mandrel form from the plurality of base layers and the skin layer.

6. The method of claim 5, wherein said removing the mandrel form involves dissolving the mandrel form using an etchant.

7. The method of claim 1, wherein the integrated, flexible diaphragm is flexible relative to the plurality of base layers.

8. The method of claim 1, wherein the integrated, flexible diaphragm has a thickness that is between 10-40 μm.

9. The method of claim 1, wherein the integrated, flexible diaphragm has a thickness that is approximately 3-15% of a combined thickness of the skin layer and the plurality of base layers.

10. The method of claim 1, wherein the integrated, flexible diaphragm comprises a plurality of corrugations.

11. The method of claim 10, wherein the plurality of corrugations comprise one or more concentric rings.

12. The method of claim 1, wherein the skin layer comprises a plurality of layers of the material.

13. The method of claim 1, wherein the skin layer comprises a superelastic nickel titanium material.

14. The method of claim 1, wherein the material of the plurality of base layers comprises superelastic nickel titanium.

15. The method of claim 1, wherein the portion of the body portion of the mandrel form surrounding the diaphragm portion is planar.

16. The method of claim 1, wherein the portion of the body portion of the mandrel form surrounding the diaphragm portion is curved.

17. A method of manufacturing an integrated, flexible diaphragm, the method comprising:
providing a mandrel form, the mandrel form including a body portion and a diaphragm portion;
placing the mandrel form in a vacuum deposition chamber;
depositing one or more first layers of material over the diaphragm portion and at least a portion of the body portion of the mandrel form surrounding the diaphragm portion to form the integrated, flexible diaphragm;
disposing a mask layer over the diaphragm; and
depositing one or more second layers of material over the masked diaphragm and at least the portion of the body portion of the mandrel form surrounding the diaphragm portion.

18. The method of claim 17, further comprising:
prior to said depositing the one or more first layers of material, masking the mandrel form with a sacrificial layer.

19. The method of claim 17, further comprising:
after said depositing the one or more second layers of material, removing the mandrel form from the one or more first layers of material and the one or more second layers of material.

20. The method of claim 19, wherein said removing the mandrel form involves dissolving the mandrel form or one or more layers thereof using an etchant.
